(19) United States
(12) Patent Application Publication
(10) Pub. No.: US 2019/0008374 A1
(43) Pub. Date: Jan. 10, 2019

(54) ENDOSCOPE SYSTEM

(71) Applicant: OLYMPUS CORPORATION, Tokyo (JP)

(72) Inventor: Yusuke Yamamoto, Kawasaki-shi (JP)

(73) Assignee: OLYMPUS CORPORATION, Tokyo (JP)

(21) Appl. No.: 16/130,023

(22) Filed: Sep. 13, 2018

Related U.S. Application Data
(63) Continuation of application No. PCT/JP2016/061554, filed on Apr. 8, 2016.

(51) Int. Cl. A61B 1/05; A61B 1/00; G02B 23/24; A61B 1/06

(52) U.S. Cl. CPC A61B 1/05; A61B 1/00009; A61B 1/045; G02B 23/2461; A61B 1/0661; A61B 1/00096

(57) ABSTRACT

An endoscope system has a light source, an imaging device, and an operation device. The imaging device has a first substrate, a second substrate, and an optical filter. The optical filter is disposed between the first substrate and the second substrate and allows only light having a predetermined wavelength band to pass the therethrough. In all wavelengths included in the predetermined wavelength band, a light absorption coefficient of oxidized hemoglobin is larger than a light absorption coefficient of reduced hemoglobin or the light absorption coefficient of the oxidized hemoglobin is smaller than the light absorption coefficient of the reduced hemoglobin. The operation device calculates an oxygen saturation on the basis of a first pixel signal generated in the first substrate and a second pixel signal generated in the second substrate.

### BACKGROUND OF THE INVENTION

### Field of the Invention

The present invention relates to an endoscope system. The present application is a continuation application based on International Patent Application No. PCT/JP2016/061554 filed on Apr. 8, 2016, the content of which is incorporated herein by reference.

### Description of Related Art

Endoscope systems capable of acquiring biological function information in addition to normal observation using visible light have attracted attention. For example, Japanese Unexamined Patent Application, First Publication No. 2014-094088 discloses an endoscope system capable of detecting the oxygen saturation level of hemoglobin in blood as biological function information. In the endoscope system disclosed in Japanese Unexamined Patent Application, First Publication No. 2014-094088, visible light and light for measuring the oxygen saturation are sequentially irradiated to an observation target. Respective reflected light of the visible light and the light for measuring the oxygen saturation are imaged by an image sensor and pixel signals are generated. On the basis of the pixel signals, the oxygen saturation in an observation region is calculated.

FIG. 14 shows a hardware configuration of an endoscope system 1001, which has a configuration similar to that disclosed in Japanese Unexamined Patent Application, First Publication No. 2014-094088. As shown in FIG. 14, the endoscope system 1001 has a light source unit 1010, an endoscope unit 1020, an operation device 1030, and a monitor 1040.

The light source unit 1010 has a first light source 1100, a second light source 1101, and a light source control section 1102. The first light source 1100 generates visible light. The second light source 1101 generates light for measuring an oxygen saturation. The light source control section 1102 controls the first light source 1100 and the second light source 1101 such that the first light source 1100 and the second light source 1101 are sequentially turned on.

FIG. 15 shows wavelength characteristics of the light generated by the second light source 1101. In FIG. 15, a horizontal axis of the graph indicates a wavelength (nm) of the light and a vertical axis of the graph indicates a light amount. The light generated by the second light source 1101 includes light having a wavelength of 473 nm.

The endoscope unit 1020 has a light guide 1200, an illumination lens 1201, an objective lens 1202, and an imager 1203. The light from the light source unit 1010 is incident on the light guide 1200. The light guide 1200 transmits the light from the light source unit 1010 to a distal end part of the endoscope unit 1020. The light transmitted by the light guide 1200 is irradiated to an object 1050 by the illumination lens 1201. In visible light observation, the visible light from the first light source 1100 is irradiated to the object 1050. In oxygen saturation measurement, the light from the second light source 1101 is irradiated to the object 1050.

At the distal end part of the endoscope mitt 1020, the objective lens 1202 is provided adjacent to the illumination lens 1201. Light reflected by the object 1050 is incident on the objective lens 1202. The objective lens 1202 forms an image of the light from the object 1050. The imager 1203 is disposed at an image forming position of the objective lens 1202. The light having passed through the objective lens 1202 is incident on the imager 1203. The imager 1203 images the light incident thereon, thereby generating pixel signals. The pixel signals generated by the imager 1203 are output to the operation device 1030.

FIG. 16 shows a pixel arrangement of the imager 1203. The imager 1203 has a plurality of pixels 1203P arranged in a matrix form. The pixels 1203P generate pixel signals based on light incident thereon. A color filter is disposed on the surface of each pixel. The plurality of pixels 1203P include R pixels Pr2, G pixels Pg2, and B pixels Pb2. In FIG. 16, the pixel 1203P shown as “R” is the R pixel Pr2. In FIG. 16, the pixel 1203P shown as “G” is the G pixel Pg2. In FIG. 16, the pixel 1203P shown as “B” is the B pixel Pb2.

The R pixel Pr2 corresponds to red. A red filter is disposed on the surface of the R pixel Pr2 to allow red light to pass therethrough. The R pixel Pr2 generates a pixel signal based on the red light. In the following description, the pixel signal generated by the R pixel Pr2 is called an R signal. The G pixel Pg2 corresponds to ween. A green filter is disposed on the surface of the G pixel Pg2 to allow green light to pass therethrough. The G pixel Pg2 generates a pixel signal based on the green light. In the following description, the pixel signal generated by the G pixel Pg2 is called a G signal. The B pixel Pb2 corresponds to blue. A blue filter is disposed on the surface, of the B pixel Pb2 to allow blue light to pass therethrough. The B pixel Pb2 generates a pixel signal based on the blue light. In the following description, the pixel signal generated by the B pixel Pb2 is called a B signal. The pixel arrangement shown in FIG. 16 is a Bayer arrangement. In the Bayer arrangement, a basic arrangement is regular and periodical in a row direction and a column direction. The basic arrangement includes one R pixel Pr2, two G pixels Pg2, and one B pixel Pb2.

The operation device 1030 generates a visible light image signal on the basis of the pixel signals generated by the imager 1203 at the time of visible light observation. The operation device 1030 calculates an oxygen saturation on the basis of the pixel signals generated by the imager 1203 at the time of oxygen saturation measurement, and generates an oxygen saturation image signal. The visible light image signal and the oxygen saturation image signal generated by the operation device 1030 are output to the monitor 1040. The monitor 1040 displays a visible light image based on the visible light image signal and an oxygen saturation image based on the oxygen saturation image signal. For example, the monitor 1040 displays the visible light image and the oxygen saturation image side by side. Alternatively, the monitor 1040 displays the visible light image and the oxygen saturation image by overlapping them. The monitor 1040 displays an oxygen saturation distribution in an observation region as an image in realtime, so that a doctor can detect a cancerous region in a low oxygen state.

FIG. 17 shows light absorption coefficients of oxidized hemoglobin and reduced hemoglobin. In FIG. 17, a horizontal axis of the graph indicates a wavelength (nm) of light and a vertical axis of the graph indicates the light absorption coefficient. A line L10 indicates the light absorption coefficient of the oxidized hemoglobin and a line L11 indicates the light absorption coefficient of the reduced hemoglobin. As shown in FIG. 15, the wavelength distribution of the light from the second light source 1101 has a peak at the wavelength of 473 nm. As shown in FIG. 17, at the wavelength of 473 nm, difference between the light absorption coefficients of the oxidized hemoglobin and the reduced hemoglobin is large. It is easy to acquire information on the oxygen saturation from pixel signals based on light having a wavelength at which the difference between the light absorption coefficients is large.

### SUMMARY OF THE INVENTION

According to a first aspect of the present invention, an endoscope system has a light source, an imaging device, and an operation device. The light source generates illumination light including visible light. The imaging device images reflected light of the illumination light irradiated to an object from the light source. The imaging device has a first substrate, a second substrate, and an optical filter. The first substrate has a plurality of first pixels. The second substrate is stacked on the first substrate and has a plurality of second pixels. The optical filter is disposed between the first substrate and the second substrate and allows only light having passed through the first substrate and having a predetermined wavelength band for calculating an oxygen saturation to pass therethrough. At all wavelengths included in the predetermined wavelength band, a light absorption coefficient of oxidized hemoglobin is larger than a light absorption coefficient of reduced hemoglobin. Alternatively, at all the wavelengths included in the predetermined wavelength band, the light absorption coefficient of the oxidized hemoglobin is smaller than the light absorption coefficient of the reduced hemoglobin. The reflected light is incident on the plurality of first pixels. The light having passed through the first substrate and the optical filter is incident on the plurality of second pixels. The first pixel included in the plurality of first pixels generates a first pixel signal based on the light incident in the first pixel. The second pixel included in the plurality of second pixels generates a second pixel signal based on the light incident on the second pixel. The operation device calculates the oxygen saturation on the basis of the first pixel signal and the second pixel signal.

According to a second aspect of the present invention, in the first aspect, the predetermined wavelength band may be a wavelength hand in which a wavelength is equal to or more than 500 nm.

According to a third aspect of the present invention, in the second aspect, the predetermined wavelength band may be included in a wavelength band in which a wavelength is 600 nm to 750 nm and may have a width equal to or less than 100 nm.

According to a fourth aspect of the present invention, in the second aspect, the light source may generate the illumination light including light having the predetermined wavelength band, in addition to the visible light. The predetermined wavelength band may be included in a wavelength band in which a wavelength is 800 nm to 900 nm and may have a width equal to or less than 100 nm.

According to a fifth aspect of the present invention, in the first aspect, the operation device may generate a corrected pixel signal by subtracting a signal based on the second pixel signal from the first pixel signal. The operation device may calculate the oxygen saturation on the basis of the corrected pixel signal and the second pixel signal.

According to a sixth aspect of the present invention, in the first aspect, the plurality of first pixels may include a G pixel that generates the first pixel signal based on green light. The operation device may calculate a blood volume in each first pixel included in the plurality of first pixels on the basis of the first pixel signal generated in the G pixel and the second pixel signal. The operation device may process the first pixel signals in order to emphasize and display a region, where the blood volume is smaller than a first threshold value, in air image based on the first pixel signals generated in the plurality of first pixels.

According to a seventh aspect of the present invention, in the sixth aspect, the operation device may process the first pixel signals in order to emphasize and display only a region, where the blood volume is smaller than the first threshold value and is equal to or more than a second threshold value, in the image based on the first pixel signals generated in the plurality of first pixels. The second threshold value is smaller than the first threshold value.

According to an eighth aspect of the present invention, in the first aspect, the operation device may calculate the oxygen saturation in each first pixel included in the plurality of first pixels and process the first pixel signals in order to emphasize and display a region, where the oxygen saturation is smaller than a predetermined threshold value, in the image based on the first pixel signals generated in the plurality of first pixels.

### BRIEF DESCRIPTION OF THE DRAWINGS

FIG. 1 is a block diagram showing a hardware configuration of an endoscope system according to a first embodiment of the present invention.

FIG. 2 is a graph shoving transmission characteristics of an optical filter according to the first embodiment of the present invention.

FIG. 3 is a sectional view of an imager according to the first embodiment of the present invention.

FIG. 4 is a graph shoving transmission characteristics of a color filter according to the first embodiment of the present invention.

FIG. 5 is a graph shoving transmission characteristics of an optical filter (an interlayer filter) according to the first embodiment of the present invention.

FIG. 6 is a reference diagram showing a pixel arrangement of the imager according to the first embodiment of the present invention.

FIG. 7 is a graph showing information stored in a memory according to the first embodiment of the present invention.

FIG. 8 is a graph showing absorption coefficients of oxidized hemoglobin and reduced hemoglobin.

FIG. 9 is a graph showing transmission characteristics of an optical filter according to a second embodiment of the present invention.

FIG. 10 is a graph showing transmission characteristics of an optical filter (an interlayer filter) according to the second embodiment of the present invention.

FIG. 11 is a reference diagram showing a pixel arrangement of an imager according to the second embodiment of the present invention.

FIG. 12 is a graph showing information stored in a memory according to the second embodiment of the present invention.

FIG. 13 is a block diagram showing a hardware configuration of an endoscope system according to a third embodiment of the present invention.

FIG. 14 is a block diagram showing a hardware configuration of an endoscope system according to the related art.

FIG. 15 is a graph showing wavelength characteristics of light generated by a light source included in the endoscope system according to the related art.

FIG. 16 is a reference diagram showing a pixel arrangement of an imager included in the endoscope system of the related art.

FIG. 17 is a graph showing absorption coefficients of oxidized hemoglobin and reduced hemoglobin.

### DETAILED DESCRIPTION OF THE INVENTION

Embodiments of the present invention will be described with reference to the drawings.

### First Embodiment

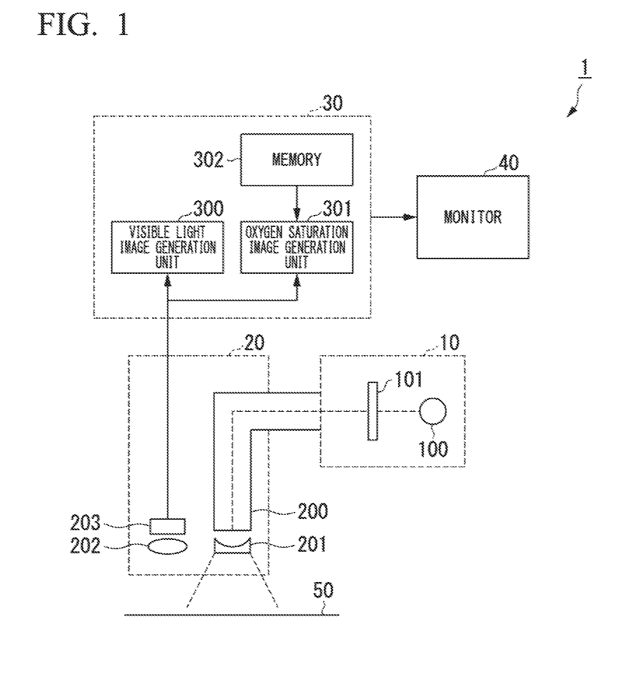

FIG. 1 shows a hardware configuration of an endoscope system 1 according to a first embodiment of the present invention. As shown in FIG. 1, the endoscope system 1 has a light source unit 10, an endoscope unit 20, an operation device 30, and a monitor 40.

The light source unit 10 has a light source 100 and an optical filter 101. The light source 100 generates illumination light including visible light. A wavelength band of the visible light includes a red wavelength band, a green wavelength band, and a blue wavelength band. The red wavelength band is a band in which a wavelength is longer than that of the green wavelength band. The green wavelength band is a band in which a wavelength is longer, than that of the blue wavelength band. The illumination light generated by the light source 100 may include light having a wavelength longer than that of the visible light, that is, infrared light.

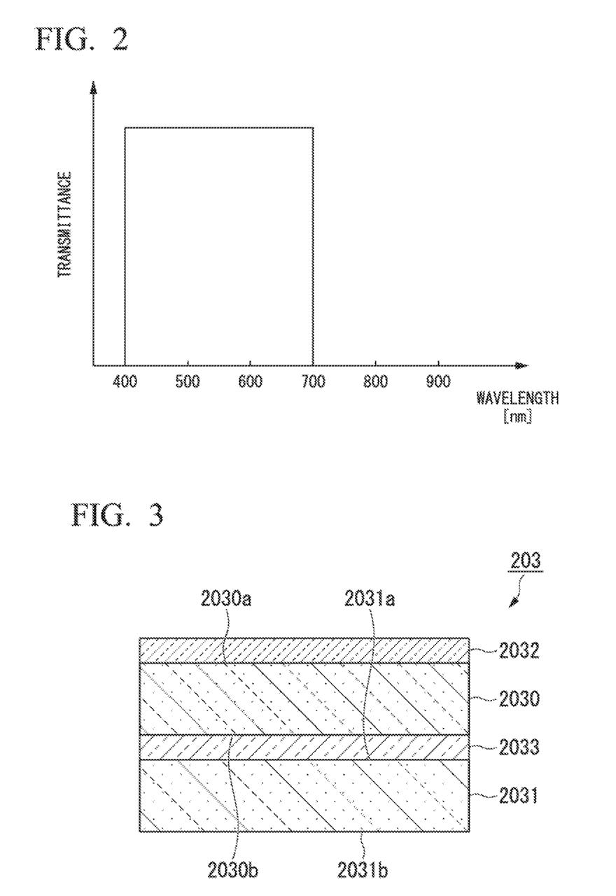

The optical filter 101 is provided on an illumination light path of the light source 100. FIG. 2 shows the transmission characteristics of the optical filter 101. In FIG. 2, a horizontal axis of the graph indicates a wavelength and a vertical axis of the graph indicates a transmittance. As shown in FIG. 2, the optical filter 101 allows only light having a wavelength band, in which a wavelength is 400 nm to 700 nm, to pass therethrough. That is, the optical filter 101 allows only the visible light included in the light from the light source 100 to pass therethrough.

The endoscope unit 20 has a light guide 200, an illumination lens 201, an objective lens 202, and an imager 203 (an image sensor). The light from the light source unit 100 is incident on the light guide 200 via the optical filter 101. The light guide 200 transmits the light from the light source unit 100 to a distal end part of the endoscope unit 20. The light transmitted by the light guide 200 is irradiated to an object 50 by the illumination lens 201. Due to the transmission characteristics of the optical filter 101, the light having the wavelength band in which a wavelength is 400 nm to 700 nm is irradiated to the object 50.

At the distal end part of the endoscope unit 20, the objective lens 202 is provided adjacent to the illumination lens 201. Light reflected by the object 50 is incident on the objective lens 202. The objective lens 202 forms an image of the light from the object 50. The imager 203 is disposed at an image forming position of the objective lens 202. The light having passed through the objective lens 202 is incident on the imager 203. Due to the transmission characteristics of the optical filter 101, the light having the wavelength band in which a wavelength is 400 nm to 700 nm is incident on the imager 203. The imager 203 images the light incident thereon, thereby generating pixel signals. The pixel signals generated by the imager 203 are output to the operation device 30.

The operation device 30 has a visible light image generation unit 300, an oxygen saturation image generation unit 301, and a memory 302. For example, the operation device 30 includes one or a plurality of processors. For example, the processor includes at least one of a central processing unit (CPU), a digital signal processor (DSP) a graphic processing unit (GPU), an application specific integrated circuit (ASIC), a field-progammable gate army (FPGA) and the like. For example, the visible image generation unit 300 and the oxygen saturation image generation unit 301 are each configured using different processors. The visible light image generation unit 300 and the oxygen saturation image generation unit 301 may be configured using the same processor.

The visible light image generation unit 300 generates a visible light image signal on the basis of the pixel signals generated by the imager 203. The visible light image signal is a signal for displaying a visible light image. The oxygen saturation image generation unit 301 calculates an oxygen saturation on the basis of the pixel signals generated by the imager 203, and generates an oxygen saturation image signal. The oxygen saturation image signal is a signal for displaying an oxygen saturation image. The oxygen saturation image is a color image on which information on the oxygen saturation is superimposed. The visible light image signal and the oxygen saturation image signal generated by the operation device 30 are output to the monitor 40. The memory 302 stores information required for calculating the oxygen saturation.

The monitor 40 displays the visible light image based on the visible light image signal and the oxygen saturation image based on the oxygen saturation image signal. For example, the monitor 40 displays the visible light image and the oxygen saturation image side by side. Alternatively, the monitor 40 displays the visible light image and the oxygen saturation image by overlapping them. The monitor 40 displays an oxygen saturation distribution in an observation region as an image in realtime, so that a doctor can detect a cancerous region in a low oxygen state.

FIG. 3 shows a configuration of the imager 203. FIG. 3 shows a section of the imager 203. As shown in FIG. 3, the imager 203 has a first substrate 2030, a second substrate 2031, a color filter 2032, and an optical filter 2033 (interlayer filter). These are stacked in a thickness direction of the first substrate 2030.

The first substrate 2030 and the second substrate 2031 are semiconductor substrates. For example, the first substrate 2030 and the second substrate 2031 are formed of silicon (Si). The first substrate 2030 has a surface 2030a and a surface 2030b. The surface 2030a and the surface 2030b are principal surfaces of the first substrate 2030. The principal surfaces are relatively large surfaces of a plurality of surfaces constituting a surface of a substrate. The surface 2030a and the surface 2030b face in opposite directions. The second substrate 2031 has a surface 2031a and a surface 2031b. The surface 2031a and the surface 2031b are principal surfaces of the second substrate 2031. The surface 2031a and the surface 20311 face in opposite directions. The surface 2030b of the first substrate 2030 and the surface 2031a of the second substrate 2031 face each other. As shown in FIG. 1, the operation device 30 is disposed outside the imager 203. At least one of the first substrate 2030 and the second substrate 2031 may include at least a part of the operation device 30.

The color filter 2032 is stacked on the surface 2030a of the first substrate 2030. The color filter 2032 has a red filter, a green filter, and a blue filter. The imager 203 need not have the color filter 2032. It is sufficient for the color filter 2032 to be disposed at any position on an optical path from the object 50 to the first substrate 2030.

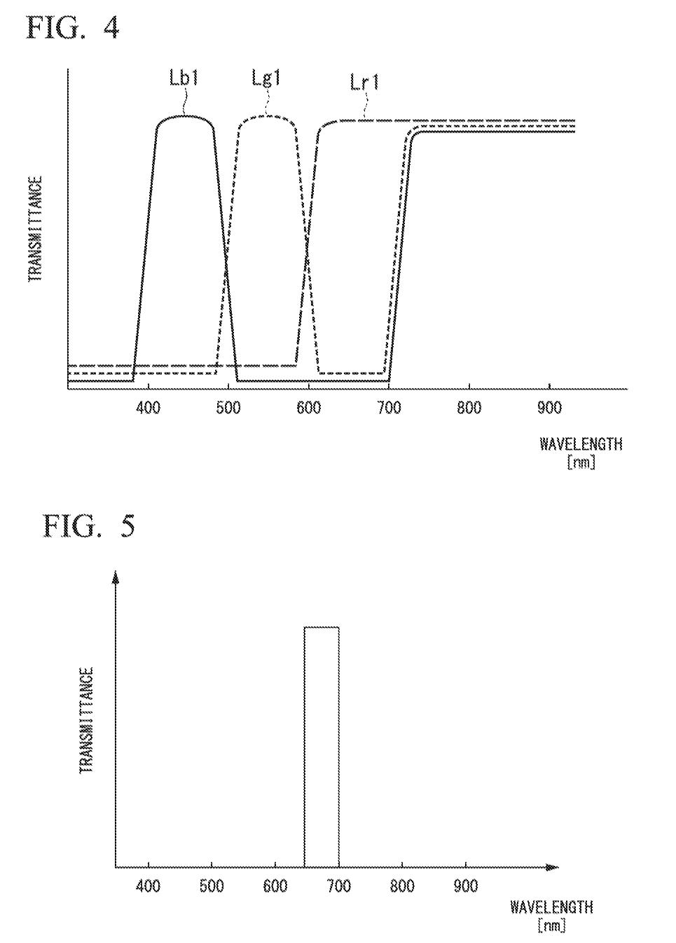

FIG. 4 shows the transmission Characteristics of the color filter 2032. In FIG. 4, a horizontal axis of the graph indicates a wavelength and a vertical axis of the graph indicates a transmittance. A line Lb1 indicates the transmission characteristics of the blue filter. As indicated by the line Lb1, the blue filter allows light having a wavelength band in which a wavelength is about 400 nm to about 500 nm and light having a wavelength band in which a wavelength is equal to or more than about 700 nm to pass therethrough. That is, the blue filter allows blue light and infrared light to pass therethrough. A line Lg1 indicates the transmission characteristics of the green filter. As indicated by the line Lg1, the green filter allows light having a wavelength band in which a wavelength is about 500 nm to about 600 nm and light having a wavelength band in which a wavelength is equal to or more than about 700 nm to pass therethrough. That is, the green filter allows green light and infrared light to pass therethrough. A line al indicates the transmission characteristics of the red filter As indicated by the line Lr1, the red filter allows light having a wavelength band in which a wavelength is equal to or more than about 600 nm to pass therethrough. That is, the red filter allows red light and infrared light to pass therethrough. Only the visible light having passed through the objective lens 202 is incident on the color filter 2032. The blue filter allows only the blue light of the visible light incident on the color filter 2032 to pass therethrough. The green filter allows only the green light of the visible light incident on the color filter 2032 to pass therethrough. The red filter allows only the red light of the visible light incident on the color filter 2032 to pass therethrough.

The optical filter 2033 is disposed between the first substrate 2030 and the second substrate 2031. FIG. 5 shows the transmission characteristics of the optical filter 2033. In FIG. 5, a horizontal axis of the graph indicates a wavelength and a vertical axis of the graph indicates a transmittance. As shown in FIG. 5, the optical filter 2033 allows only light having a wavelength band in which a wavelength is about 650 nm to about 700 nm to pass therethrough. That is, the optical filter 2033 allows only red light to pass therethrough.

Each of the first substrate 2030 and the second substrate 2031 has a plurality of pixels. Each of the plurality of pixels has a photoelectric conversion element (a photodiode) and a signal reading circuit. The photoelectric conversion element converts light incident on a pixel into a signal. The signal reading circuit reads the signal from the photoelectric conversion element and outputs the read signal as a pixel signal. The pixels of the first substrate 2030 generate a first pixel signal. The pixels of the second substrate 2031 generate a second pixel signal. The first pixel signal and the second pixel signal are output to the operation device 30.

The light having passed through the color filter 2032 is incident on the surface 2030a of the first substrate 2030. The first substrate 2030 is a backside irradiation type imaging substrate. For example, a thickness of the first substrate 2030 is several μm. That is, the first substrate 2030 is thin. The light absorptivity of silicon differs depending on the wavelength of light. The light absorptivity of silicon for light having a short wavelength is high. The light absorptivity of silicon for light having a long wavelength is low. Therefore, light having a shorter wavelength is likely to be absorbed at a shallower position in the silicon. Light having a longer wavelength is likely to be absorbed at a deeper position in the silicon. For example, when the thickens of the first substrate 2030 is 3 μm, a part of light having a wavelength equal to or more than 500 nm is not absorbed in the first substrate 2030 and passes through the first substrate 2030. That is, light of a band on a high wavelength side of a green wavelength band and red light pass through the first substrate 2030.

The light having passed through the first substrate 2030 is incident on the optical filter 2033. Light having a wavelength equal to or more than 500 nm is incident on the optical filter 2033. A wavelength of light having passed through the optical filter 2033 is 650 nm to 700 nm. The light having passed through the optical filter 2033 is incident on the surface 2031a of the second substrate 2031.

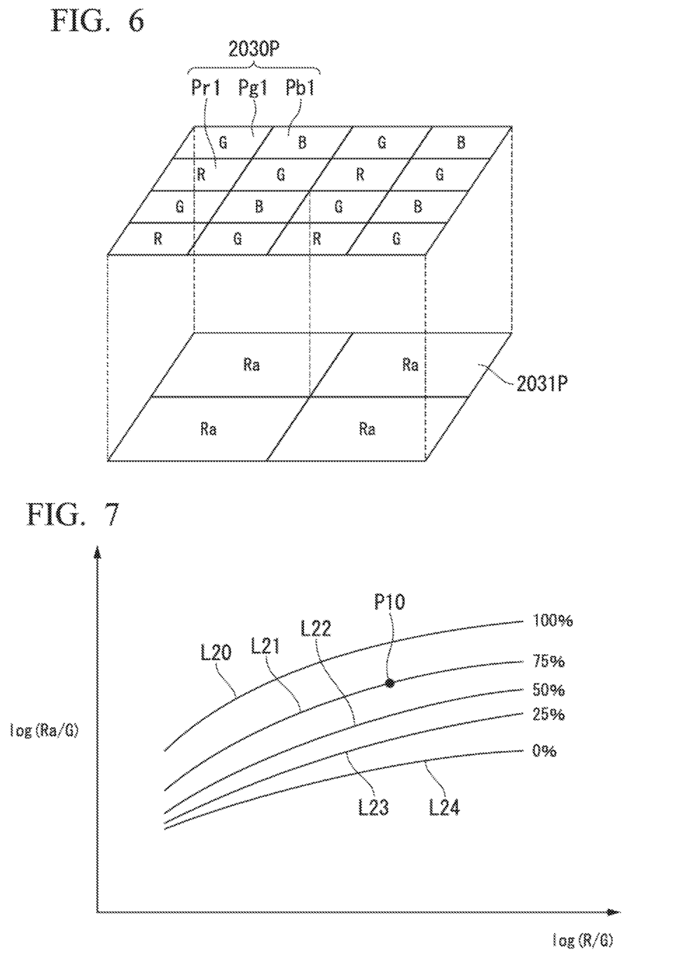

FIG. 6 shows a pixel arrangement of the imager 203. The first substrate 2030 of the imager 203 has a plurality of first pixels 2030P arranged in a matrix form. The light, which has passed through the color filter 2032, included in the light from the object 50 is incident on the plurality of first pixels 2030P The first pixels 2030P generate a first pixel signal based on the light incident thereon. The plurality of first pixels 2030P include R pixels Pr1, G pixels Pg1, and B pixels Pb1. In FIG. 6, the first pixel 2030P shown as “R” is the R pixel Pr1. In FIG. 6, the first pixel 2030P shown as “G” is the G pixel Pg1. In FIG. 6, the first pixel 2030P shown as “B” is the B pixel Pb1.

The R pixel Pr1 corresponds to red. The red filter is disposed on the surface of the R pixel Pr1. The R pixel PR1 generates the first pixel signal based on red light. In the following description, the first pixel signal generated by the R pixel Pr1 is called air R signal. The G pixel Pg1 corresponds to green. The green filter is disposed on the surface of the G pixel Pg1. The G pixel Pg1 generates the first pixel signal based on gem light. In the following description, the first pixel signal generated by the G pixel Pg1 is called a G signal. The B pixel Pb1 corresponds to blue. The blue filter is disposed on the surface of the B pixel Pb1. The B pixel Pb1 generates the first pixel signal based on blue light. In the following description, the first pixel signal generated by the B pixel Pb1 is called a B signal. The pixel arrangement of the plurality of first pixels 2030P shown in FIG. 6 is a Bayer arrangement. In the Bayer arrangement, a basic arrangement is regular and periodical in a row direction and a column direction. The basic arrangement includes one R pixel Pr1, two G pixels Pg1, and one B pixel Pb1.

The second substrate 2031 of the imager 203 has a plurality of second pixels 2031P arranged in a matrix form. One second pixel 2031P is disposed in a region corresponding to four first pixels 2030P. The four first pixels 2030P corresponding to one second pixel 2031P constitute the basic arrangement of the Bayer arrangement. Light having passed through each of the four first pixels 2030P and the optical filter 21333 is incident on one second pixel 203IR The second pixel 2031P generates a second pixel signal based an the light incident on the second pixel 2031P. In the following description, the second pixel signal generated by the second pixel 2031P is called an Ra signal.

Since the first substrate 2030 and the second substrate 2031 are stacked, the first pixels 2030P and the second pixels 2031P can simultaneously detect light. Therefore, the first pixel signal and the second pixel signal are less likely to be affected by the movement of the object 50 or the endoscope unit 20. Consequently, as compared with the case where the visible light observation and the oxygen saturation measurement are sequentially performed, the accuracy of calculation of the oxygen saturation is improved.

The operation device 30 (the visible light image generation unit 300) generates the visible light image signal on the basis of the first pixel signal (the R signal, the G signal, and the B signal) generated in the first pixels 2030P. The operation device 30 (the oxygen saturation image generation unit 301) generates the oxygen saturation image signal on the basis of the first pixel signal (the R signal and the G signal) generated in the first pixels 2030P and the second pixel signal (the Ra signal) generated in the second pixels 2031P.

A calculation method of the saturation will be described. The operation device 30 (the oxygen saturation image generation unit 301) calculates a first intensity ratio and a second intensity ratio for each of the plurality of first pixels 2030P. The first intensity ratio is an intensity ratio (Ra/G) of the Ra signal and the G signal. The second intensity ratio is an intensity ratio (R/G) of the R signal and the G signal. The Ra signal generated in one second pixel 2031P is used to calculate the first intensity ratio of four first pixels 2030P corresponding to the second pixel 2031P. G signal of the R pixel Pr1 is interpolated on the basis of the G signal of the pixels Pg1 around the R pixel Pr1. B signal of the R pixel Pr1 is interpolated on the basis of the B signal of the B pixels Pb1 around the R pixel Pr1. R signal of the G pixel Pg1 is interpolated on the basis of the R signal of the R pixels Pr1 around the G pixel Pg1. B signal of the G pixel Pg1 is interpolated on the basis of the B signal of the B pixels Pb1 around the G pixel Pg1. R signal of the B pixel Pb1 is interpolated on the basis of the R signal of the R pixels Pr1 around the G pixel Pg1. G signal of the B pixel Pb1 is interpolated on the basis of the G signal of the G pixels Pg1 around the B pixel Pb1.

The memory 302 stores information indicating a correlation among the first intensity ratio, the second intensity ratio, and the oxygen saturation. FIG. 7 shows the information stored in the memory 302. In the graph shown in FIG. 7, a horizontal axis and a vertical axis indicate a logarithmic scale. In FIG. 7, the horizontal axis of the graph indicates log (R/G) and the vertical axis of the graph indicates log (Ra/G). Five curves L20, L21, L22, L23, and L24 shown in FIG. 7 indicate a correlation between the first intensity ratio and the second intensity ratio corresponding to oxygen saturations different from one another. The curve L20 shows a correlation between the first intensity ratio and the second intensity ratio when the oxygen saturation is 100%. The curve L21 shows a correlation between the first intensity ratio and the second intensity ratio when the oxygen saturation is 75%. The curve L22 shows a correlation between the first intensity ratio and the second intensity ratio when the oxygen saturation is 50%. The curve L23 shows a correlation between the first intensity ratio and the second intensity ratio when the oxygen saturation is 25%. The curve L24 shows a correlation between the first intensity ratio and the second intensity ratio when the oxygen saturation is 0%. The curves L20, L21, L22, L23, and L24 shown in FIG. 7 are acquired on the basis of a light scattering simulation. In the light scattering simulation, light absorption coefficients of oxidized hemoglobin and reduced hemoglobin, a light scattering coefficient of a living body, and the like are considered.

The operation device 30 (the oxygen saturation image generation unit 301) reads the information of the graph shown in FIG. 7 from the memory 302. The operation device 30 (the oxygen saturation image generation unit 301) collates the calculated first intensity ratio and second intensity ratio and the information read from the memory 302 On the basis of a positional relationship between points corresponding to the first intensity ratio and the second intensity ratio in the first pixels 2030P and the curves L20, L21, L22, L23, and L24, the oxygen saturations are decided. In FIG. 7, a point P10 corresponding to the first intensity ratio and the second intensity ratio in a certain first pixel 2030P coincides with a point on the curve L21. The curve L21 corresponds to the oxygen saturation of 75%. Therefore, the oxygen saturation corresponding to the certain first pixel 2030P is decided to be 75%. The oxygen saturation is calculated for each of the plurality of first pixels 2030P. The oxygen saturation may be calculated for only the first pixels 2030P corresponding to a vessel region in an angle of view. The vessel region can be specified by the R signal, a ratio of the R signal and the B signal, and the like.

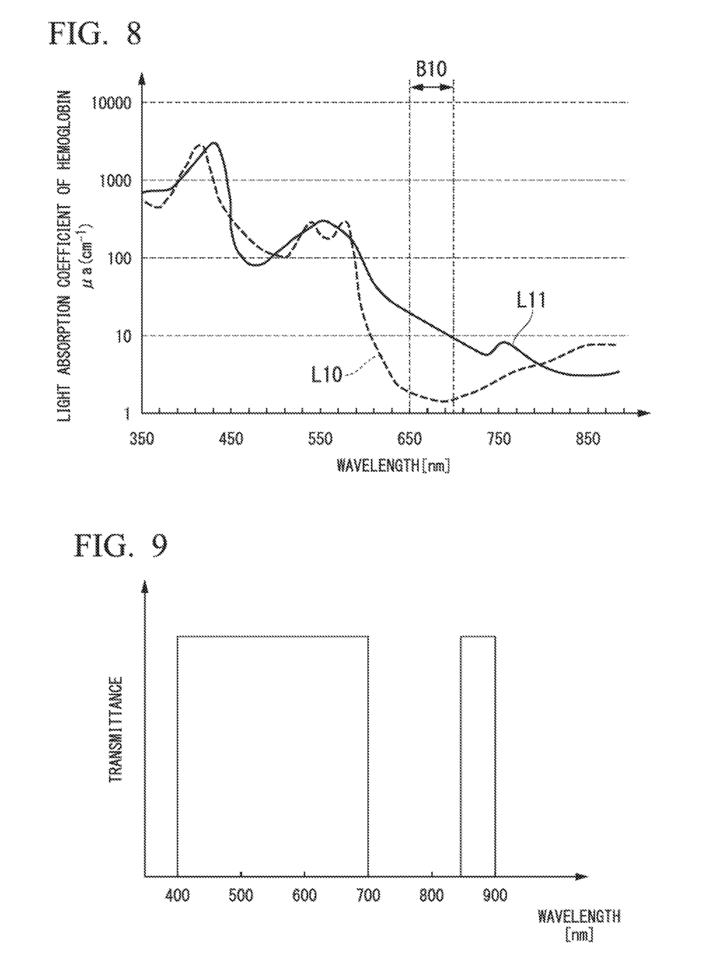

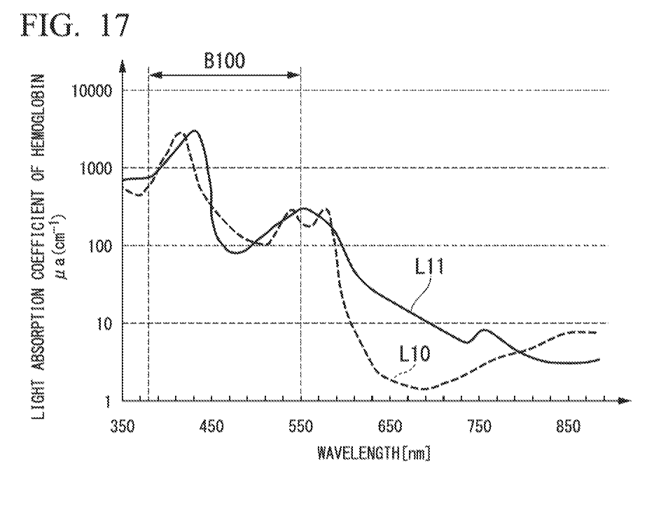

The correlation among the first intensity ratio, the second intensify ratio, and the oxygen saturation are closely associated with light absorption characteristics of hemoglobin. FIG. 8 shows the light absorption coefficients of the oxidized hemoglobin and the reduced hemoglobin. In FIG. 8, a horizontal axis of the graph indicates a wavelength (urn) of light and a vertical axis of the graph indicates light absorption coefficients. A line L10 indicates the light absorption coefficient of the oxidized hemoglobin and a line L11 indicates the light absorption coefficient of the reduced hemoglobin. A wavelength band B10 is a wavelength band (650 nm to 700 nm) of light passing through the optical filter 2033. In all wavelengths in the wavelength band B10 of the light passing through the optical filter 2033, the light absorption coefficient of the oxidized hemoglobin is smaller than the light absorption coefficient of the reduced hemoglobin. Therefore, as compared with the case of calculating the oxygen saturation by using the pixel signals based on the light of the wavelength band B100 in FIG. 17, the accuracy of calculation of the oxygen saturation is improved.

In the wavelength band B10 of the light passing through the optical filter 2033, since the difference between the light absorption coefficients of the oxidized hemoglobin and the reduced hemoglobin is large, it is easy to acquire information on the oxygen saturation from the pixel signals. However, the Ra signal corresponding to the light of the wavelength band B10 is changed depending on the oxygen saturation and a blood volume. The R signal is mainly changed depending on the blood volume. The G signal is a reference signal (a standardized signal) of the Ra signal and the R signal. The operation device 30 (the oxygen saturation image generation unit 301) can calculate the oxygen saturation, regardless of the blood volume, by using the first intensity ratio (Ra/G) and the second intensity ratio (R/G) obtained from the Ra signal, the R signal, and the G signal.

The operation device 30 (the oxygen saturation image generation unit 301) generates the oxygen saturation image signal on the basis of the R signal, the G the B signal, and the oxygen saturation. For example, the operation device 30 (the oxygen saturation image generation unit 301) multiplies only the B signal by a gain larger than 1 in the first pixel 2030P in which the oxygen saturation is smaller than a reference value γ. The reference value γ is a threshold value for determining a region where the oxygen saturation is small. For example, the reference value γ is 60%. The reference value γ may be a value which can be designated by a user. Instead of multiplying only the B signal by the gain, a gain may be multiplied to the R signal the G signal and the B signal and the gain multiplied to the B signal may be larger than a gain multiplied to the R signal and the G signal. The oxygen saturation image signal includes the R signal, the G signal, and the B signal of each first pixel 2030P.

In a region where the oxygen saturation is smaller than the reference value γ, since the gain multiplied to the B signal is large, the region has a bluish color in the oxygen saturation image. In general, in a color in a living body, a red component is large and a blue component is small. Therefore, the bluish region is conspicuous in the oxygen saturation image. In this way, a doctor can easily detect a cancerous region in a low oxygen state. On the other hand, in the first pixel 2030P in which the oxygen saturation is equal to or more than the reference value γ, no gain is multiplied to the B signal, the G signal, and the R signal. In the oxygen saturation image, there is no change in color tone of the region where the oxygen saturation is equal to or more than the reference value γ.

As shown in FIG. 5, the optical filter 2033 allows only light having the wavelength band in which a wavelength is 650 nm to 700 nm to pass therethrough. The wavelength band of the light passing through the optical filter 2033 is not limited to the example shown in FIG. 5. It is sufficient if the wavelength band of the light passing through the optical filter 2033 is a wavelength band in which a relationship between the sizes of the light absorption coefficients of the oxidized hemoglobin and the reduced hemoglobin is constant.

As described above, the endoscope system 1 has the light source 100, the imager 203 (an imaging device), and the operation device 30. The light source 100 generates the illumination light including the visible light. The imager 203 images the reflected light of the illumination light irradiated to the object 50 from the light source 100. The imager 203 has the first substrate 2030, the second substrate 2031, and the optical filter 2033. The first substrate 2030 has the plurality of first pixels 2030P. The second substrate 2031 is stacked on the first substrate 2030 and has the plurality of second pixels 2031P. The optical filter 2033 is disposed between the first substrate 2030 and the second substrate 2031, and allows only light, which has passed through the first substrate 2030 and has a predetermined wavelength band for oxygen saturation calculation, to pass therethrough. In all wavelengths included in the predetermined wavelength band, the light absorption coefficient of the oxidized hemoglobin is larger than the light absorption coefficient of the reduced hemoglobin. Alternatively in all the wavelengths included in the predetermined wavelength band, the light absorption coefficient of the oxidized hemoglobin is smaller than the light absorption coefficient of the reduced hemoglobin. The reflected light of the illumination light is incident on the plurality of first pixels 2030P. Light having passed through the first substrate 2030 and the optical filter 2033 is incident on the plurality of second pixels 2031P. The plurality of first pixels 2030P generate the first pixel signals based on the light incident thereon. The plurality of second pixels 2031P generates the second pixel signals based on the light incident thereon. The operation device 30 calculates the, oxygen saturation on the basis of the first pixel signals and the second pixel signals.

The endoscope system of each aspect of the present invention need not have configurations other than the configurations corresponding to the light source 100, the imager 203, and the operation device 30. For example, since the generation of the visible light image is not essential, the endoscope system of each aspect of the present invention need not have a configuration corresponding to the visible light image generation unit 300. Since the monitor 40 need not be a device subordinate to the endoscope system 1, the endoscope system of each aspect of the present invention need not have a configuration corresponding to the monitor 40.

Since the first substrate 2030 and the second substrate 2031 are stacked, the plurality of first pixels 2030P and the plurality of second pixels 2031P can simultaneously detect light. In all the wavelengths included in the predetermined wavelength band, the light absorption coefficient of the oxidized hemoglobin is larger than the light absorption coefficient of the reduced hemoglobin. Alternatively, in ah the wavelengths included in the predetermined wavelength band, the light absorption coefficient of the oxidized hemoglobin is smaller than the light absorption coefficient of the reduced hemoglobin. Therefore, the second pixel 2031P can generate the second pixel signal on the basis of light of the wavelength band in which the relationship between the sizes of the light absorption coefficients of the oxidized hemoglobin and the reduced hemoglobin is constant. Consequently the endoscope system 1 can calculate the oxygen saturation more accurately.

The predetermined wavelength band may be a wavelength band in which a wavelength is equal to or more than 500 nm. Light having the wavelength band in which a wavelength is equal to or more than 500 nm easily passes through the first substrate 2030.

The predetermined wavelength band may be included in a wavelength band in which a wavelength is 600 nm to 750 nm and may have a width equal to or less than 100 nm. As shown in FIG. 8, since the difference between the light absorption coefficients of the oxidized hemoglobin and the reduced hemoglobin is large in the wavelength band in which a wavelength is 600 nm to 750 nm, it is easy to acquire information on the oxygen saturation from the pixel signals. Since the width of the predetermined wavelength band is narrow, there is a small change in the ratio of the light absorption coefficients of the oxidized hemoglobin and the reduced hemoglobin according to a wavelength. Therefore, the endoscope system 1 can calculate the oxygen saturation with high accuracy. Infrared light is not included in the wavelength band in which a wavelength is 600 nm to 750 nm. Therefore, the endoscope system 1 can generate the visible light image signal on the basis of the first pixel signal generated in the first pixel 2030P.

The operation device 30 (the oxygen saturation image generation unit 301) may process the first pixel signal in order to emphasize and display a region, where the oxygen saturation is smaller than a predetermined threshold value (the reference value γ), in an image based on the first pixel signal generated in the plurality of first pixels 2030P. For example, the operation device 30 (the oxygen saturation image generation unit 301) increases the gain of the B signal, which is generated in the first pixel 2030P included in the region where the oxygen saturation is smaller than the reference value γ, of the first pixel signal generated in the first pixel 2030P. In this way, in the image based on the first pixel signal, the region, where the oxygen saturation is smaller than the threshold value, is conspicuous.

### Second Embodiment

A second embodiment of the present invention will be described using the endoscope system 1 shown in FIG. 1. Hereinafter, a difference from the description in the first embodiment will be mainly described. The optical filter 101 of the first embodiment allows only the visible light to pass therethrough. On the other band, the optical filter 101 of the second embodiment allows the visible light and the infrared light to pass therethrough. The optical filter 2033 of the first embodiment allows only the red light to pass therethrough. On the other hand, the optical filter 2033 of the second embodiment allows only the infrared light to pass therethrough. When the transmission characteristics of the optical filter 101 and the optical filter 2033 are changed, signal processing conducted by the visible light image generation unit 300 and the oxygen saturation image generation unit 301 are changed.

FIG. 9 shows the transmission characteristics of the optical filter 101. In FIG. 9, a horizontal axis of the graph indicates a wavelength and a vertical axis of the graph indicates a transmittance. As shown in FIG. 9, the optical filter 101 allows light having a wavelength band in which a wavelength is 400 nm to 700 nm and light having a wavelength band in which a wavelength is 850 nm to 900 nm to pass therethrough. That is, the optical filter 101 allows only the visible light and the infrared light included in the light from the light source 100 to pass therethrough. Due to the transmission characteristics of the optical filter 101, the, light having the wavelength band in which a wavelength is 400 nm to 700 nm and the light having the wavelength band in which a wavelength is 850 nm to 900 nm are incident on the imager 203.

The transmission characteristics of the color filter 2032 are the same as those shown in FIG. 4. The blue filter allows only the blue light and the infrared light included in the light incident on the color filter 2032 to pass therethrough. The green filter allows only the green light and the infrared light included in the light incident on the color filter 2032 to pass therethrough. The red filter allows only the red light and the infrared light included in the light incident on the color filter 2032 to pass therethrough.

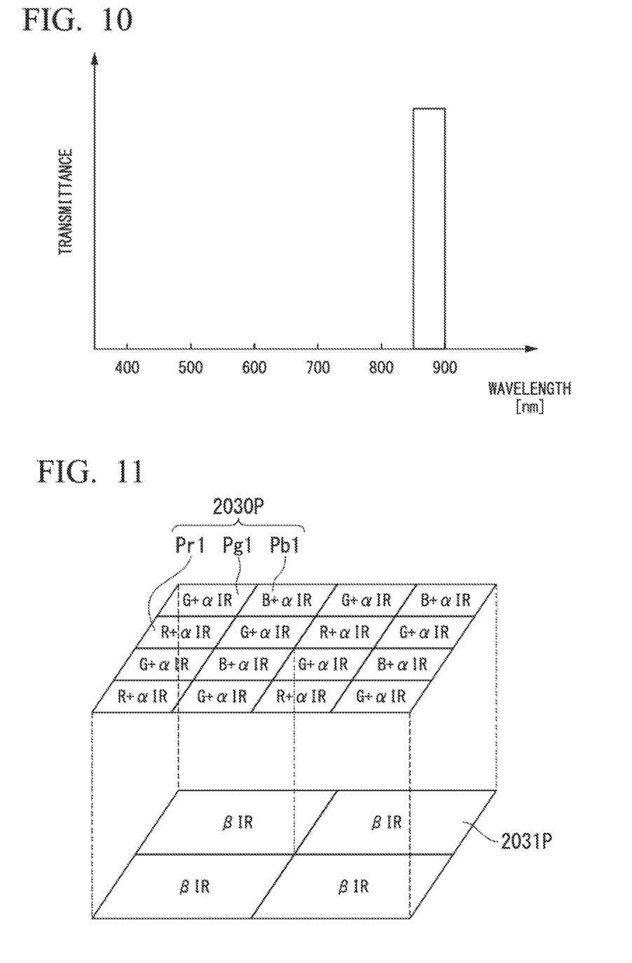

FIG. 10 shows the transmission characteristics of the optical filter 2033. In FIG. 10, a horizontal axis of the graph indicates a wavelength and a vertical axis of the graph indicates a transmittance. As shown in FIG. 10, the optical filter 2033 Allows only the light having the wavelength band in which a wavelength is 850 nm to 900 nm to pass therethrough. That is, the optical filter 2033 allows only the infrared light to pass therethrough.

In all wavelengths included in the wavelength band in which a wavelength is 850 nm to 900 nm, the light absorption coefficient of the oxidized hemoglobin is larger than the light absorption coefficient of the, reduced hemoglobin and the difference between the light absorption coefficients of the oxidized hemoglobin and the reduced hemoglobin is large. Therefore, the operation device 30 (the oxygen saturation image generation unit 301) can calculate the oxygen saturation with high accuracy.

FIG. 11 shows a pixel arrangement of the imager 203. In FIG. 11, a difference from FIG. 6 will be described. Light, which has passed through the color filter 2032, included in light from the object 50 is incident on the plurality of first pixels 2030P. The visible light and the inflated light are incident on the first pixel 2030P. A first pixel signal generated by the first pixel 2030P is different from the first pixel signal in the first embodiment. The first pixel signal includes a component based on the infrared light, in addition to a component based on the visible light. A part of the infrared light incident on the first substrate 2030 is absorbed in the first substrate 2030. Of the infrared light incident on the first substrate 2030, light, other than the light absorbed in the first substrate 2030, passes through the first substrate 2030. The infrared light having passed through the first substrate 2030 is incident on the second pixel 2031P. A second pixel signal generated by the second pixel 2031P is different from the second pixel signal in the first embodiment. The second pixel signal includes a component based on only the infrared light.

In the following description, a indicates a rate by which the first substrate 2030 absorbs the infrared light and β indicates a rate by which the second substrate 2031 absorbs the infrared light. The α and the β can be calculated from the spectral sensitivity of the first substrate 2030 and the second substrate 2031 respect to the infrared light. The α and the β are parameters based on the manufacturing conditions of the imager 203. For example, the manufacturing conditions include the thickness in an optical axis direction of each of the first substrate 2030 and the second substrate 2031. Alternatively, the manufacturing conditions include the transmission characteristics of the color filter 2032 and the optical filter 2033. The α and the β are real numbers equal to or more than 0 and equal to or less than 1.

An R pixel Pr1 generates the first pixel signal based on the red light and the infrared light, that is, an R signal. In the following description, a signal value of the R signal generated by the R pixel Pr1 is (R+αIR). A G pixel Pg1 generates the first pixel signal based on the green light and the infrared light, that is, a G signal. In the following description, a signal value of the G signal generated by the G pixel Pg1 (G+αIR). A B pixel Pill generates the first pixel signal based on the blue light and the infrared light, that is, a B signal. In the following description, a signal value of the B signal generated by the B pixel Pb1 is (B+αIR). The R indicates a signal value based on the red light. The G indicates a signal value based on the green light. The B indicates a signal value based on the blue light. The αIR indicates a signal value based on the infrared light.

The second pixel 2031P generates the second pixel signal based on the infrared light. In the following description, the second pixel signal generated by the second pixel 2031P is called an IR signal. In the following, description, a signal value of the IR signal generated by the second pixel 2031P is βIR. The βIR indicates a signal value based on the infrared light.

The operation device 30 (the visible light image generation unit 300) generates the visible light image signal on the basis of the first pixel signal (the R signal, the G signal, and the B signal) generated in the first pixel 2030P and the second pixel signal (the IR signal) generated in the second pixel 2031P The operation device 30 (the oxygen saturation image generation unit 301) generates the oxygen saturation image signal on the basis of the first pixel signal (the R signal and the G signal) generated in the first pixel 2030P and the second pixel signal (the IR signal) generated in the second pixel 2031P.

The operation device 30 (the visible light image generation unit 300) multiplies the value (that is, βIR) of the IR signal generated by the second pixel 2031P by the ratio (α/β) of α and β. The (α/β) is a coefficient based on a ratio of the rate by which the first substrate 2030 absorbs the infrared light and the rate by which the second substrate 2031 absorbs the infrared light. In this way, the operation device 30 (the visible light image generation unit 300) can calculate the signal value (αIR) based on the infrared light detected by the first pixel 2030P. The operation device 30 (the visible light image generation unit 300) subtracts the signal value (αIR) calculated by the aforementioned method from the value (R+αIR) of the R signal generated by the R pixel Pr1. In this way, the operation device 30 (the visible light image generation unit 300) generates an R signal based on only the red light. The signal value of the R signal after the subtraction is R. Similarly, the operation device 30 (the visible light image generation unit 300) subtracts the signal value (αIR) calculated by the aforementioned method from the value (G±αIR) of the G signal generated by the G pixel Pg1. In this way, the operation device 30 (the visible light image generation unit 300) generates a G signal based on only the green light. The signal value of the G signal after the subtraction is G. Similarly, the operation device 30 (the visible light image generation unit 300) subtracts the signal value (αIR) calculated by the aforementioned method from the value (β+αIR) of the B signal generated by the B pixel Pb1. In this way, the operation device 30 (the visible light image generation unit 300) generates a B signal based on only the blue light. The signal value of the B signal after the subtraction is B. The operation device 30 (the visible light image generation unit 300) generates the visible light image signal on the basis of the R signal, the G signal, and the B signal based on only the component of the visible light.

The operation device 30 (the oxygen saturation image generation unit 301) generates the R signal and the G signal based on the component of the visible light by the aforementioned method. The aforementioned operation results obtained by the visible light image generation unit 300 may be output to the oxygen saturation image generation unit 301 and the oxygen saturation image generation unit 301 may use the operation results. Alternatively the aforementioned operation results obtained by the oxygen saturation image generation unit 301 may be output to the visible light image generation unit 300 and the visible light image generation unit 300 may use the operation results. The operation device 30 (the oxygen saturation image generation unit 301) calculates a first intensity ratio and a second intensity ratio for each of the plurality of first pixels 2030P. The first intensity ratio is air intensity ratio (βIR/G) of the IR signal and the G signal. The second intensity ratio is an intensity ratio (R/G) of the R signal and the G signal. The IR signal generated in one second pixel 2031P is used to calculate the first intensity ratio of four first pixels 2030P corresponding to the second pixel 2031P.

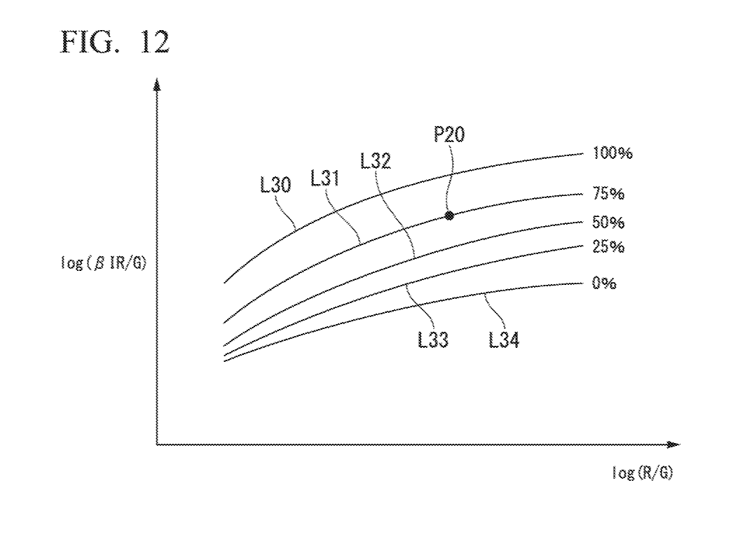

The memory 302 stores information indicating a correlation among the first intensity ratio, the second intensity ratio, and the oxygen saturation. FIG. 12 shows the information stored in the memory 302. In the graph shown in FIG. 12, a horizontal axis and a vertical axis indicate a logarithmic scale. In FIG. 12, the horizontal axis of the graph indicates log (R/G) and the vertical axis of the graph indicates log (βIR/G). Five curves L30, L31, L32, L33, and L34 shown in FIG. 12 indicate a correlation between the first intensity ratio and the second intensity ratio corresponding to oxygen saturations different from one another. The curve L30 shows a correlation between the first intensity ratio and the second intensity ratio when the oxygen saturation is 100%. The curve L31 shows a correlation between the first intensity ratio and the second intensity ratio when the oxygen saturation is 75%. The curve L32 shows a correlation between the first intensity ratio and the second intensity ratio when the oxygen saturation is 50%. The curve L33 shows a correlation between the first intensity ratio and the second intensity ratio when the oxygen saturation is 25%. The curve L34 shows a correlation between the first intensity ratio and the second intensity ratio when the oxygen saturation is 0%. The curves L30, L31, L32, L33, and L34 shown in FIG. 12 are acquired on the basis of a light scattering simulation. In the light scattering simulation, light absorption coefficients of oxidized hemoglobin and reduced hemoglobin, a light scattering, coefficient of a living body, and the like are considered.

The operation device 30 (the oxygen saturation image generation unit 301) reads the information of the graph shown in FIG. 12 from the memory 302. The operation device 30 (the oxygen saturation image generation unit 301) collates the calculated first intensity ratio and second intensity ratio and the information read from the memory 302 On the basis of a positional relationship between points corresponding to the first intensity ratio and the second intensity ratio in the first pixels 2030P and the curves L30, L31, L32, L33, and L34, the oxygen saturations are decided. In FIG. 12, a point P20 corresponding to the first intensity ratio and the second intensity ratio in a certain first pixel 2030P coincides with a point on the curve L31. The curve L31 corresponds to the oxygen saturation of 75%. Therefore, the oxygen saturation corresponding to the certain first pixel 2030P is decided to be 75%. The oxygen saturation is calculated for each of the plurality of first pixels 2030P. The oxygen saturation may be calculated for only the first pixels 2030P corresponding to a vessel region in an angle of view.

The light source 100 of the second embodiment generates the illumination light including light having a predetermined wavelength band for the oxygen saturation calculation, in addition to the visible light. The predetermined wavelength hand may be included in a wavelength band in which a wavelength is 800 nm and 900 nm and may have a width equal to or less than 100 nm.

As shown in FIG. 9 and FIG. 10, in a wavelength band in which a wavelength is equal to or more than 800 nm, a first wavelength band of light passing through the optical filter 101 is the same as a second wavelength band of light passing through the optical filter 2033. That is, in the wavelength band in which a wavelength is equal to or more than 800 nm, a first wavelength band of light incident on the imager 203 is the same as the second wavelength band of the light passing through the optical filter 2033. In the wavelength band in which a wavelength is equal to or more than 800 nm, the fist wavelength band may be eider than the second wavelength band. When the first wavelength band is wider than the second wavelength band, a component based on the infrared light in the first pixel signal includes a component based on infrared light having a wavelength different from that the infrared light incident on the second pixel 2031P. Therefore, the R signal generated by the process for subtracting the signal value (αIR) calculated on the basis of the IR signal from the value (R+αIR) of the IR signal includes a component based on infrared light which is not detectable by the second pixel 2031P. When the first wavelength band is the same as the second wavelength band, the operation device 30 (the oxygen saturation image generation unit 301) can remove a component based on the infrared light from the R signal with high accuracy. The same is applied to the G signal and the B signal.

As described above, the operation device 30 the oxygen saturation image generation unit 301) subtracts the signal (αIR) based on the second pixel signal (βIR) from the first pixel signal (for example, R+αIR), thereby generating a corrected pixel signal For example, the corrected pixel signal is an R signal having a signal value of R. The operation device 30 (the oxygen saturation image generation unit 301) calculates the oxygen saturation on the basis of the corrected pixel signal and the second pixel signal.

In the endoscope system according to the second embodiment, it is possible to use the visible light and light other than the visible light, that is, the infrared light. The endoscope system 1 according to the second embodiment can generate the oxygen saturation image signal and the visible light image signal on the basis of the first pixel signal based on the visible light and the infrared light and the second pixel signal based on the infrared light.

### Third Embodiment

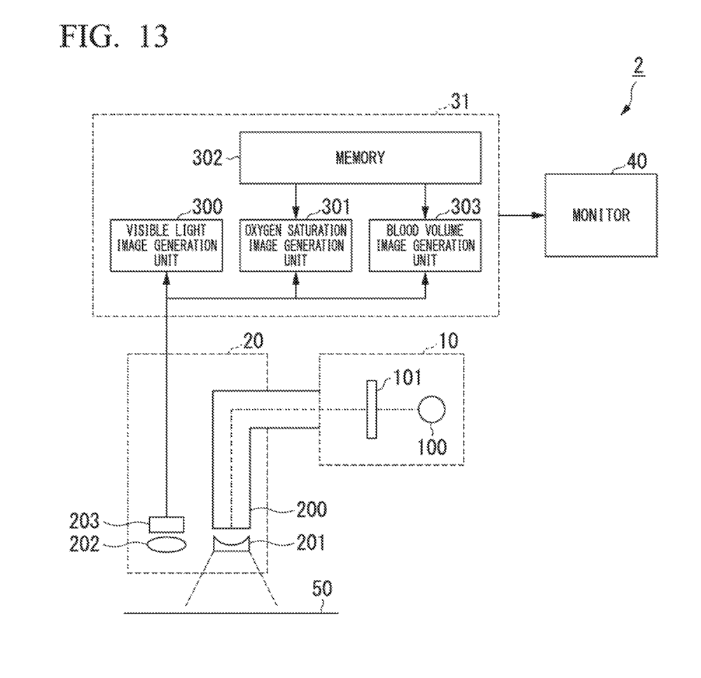

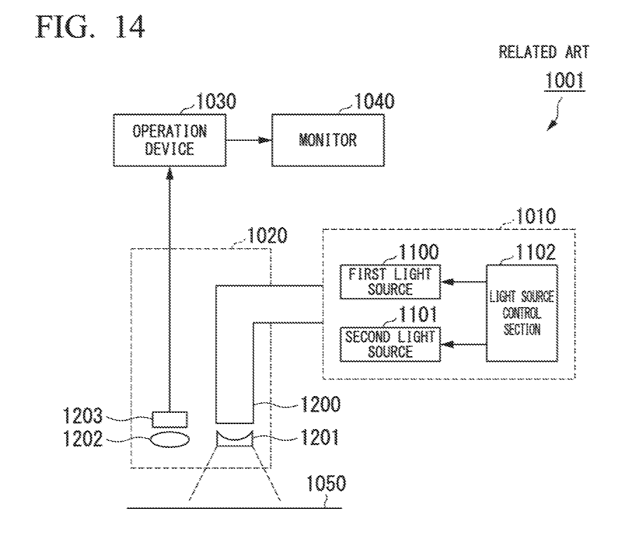

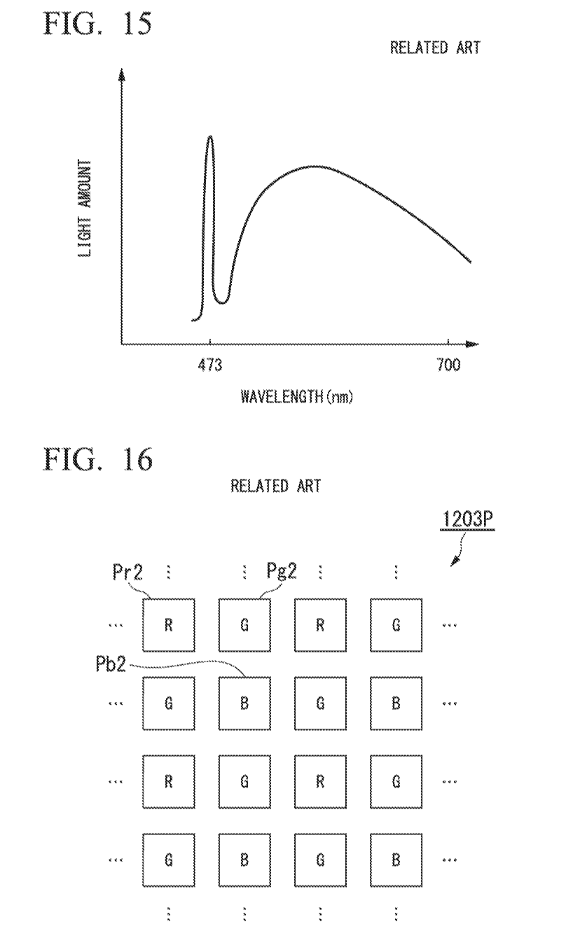

FIG. 13 shows a hardware configuration of an endoscope system 2 according to a third embodiment of the present invention. The configuration shown in FIG. 13 will be described while focusing on the difference from the configuration shown in FIG. 1.

In the endoscope system the operation device 30 in the endoscope system 1 shown in FIG. 1 is changed to an operation device 31. The operation device 31 has a blood volume image generation unit 303 in addition to the elements of the operation device 30. For example, the visible light image generation unit 300, the oxygen saturation image generation unit 301, and the blood volume image generation unit 303 are configured using processors different from one another. At least two of the visible light image generation unit 300, the oxygen saturation image generation unit 301, and the blood volume image generation unit 303 may be configured using the same processor. The blood volume image generation unit 303 calculates a blood volume on the basis of the first pixel signal (the G signal) generated in the first pixels 2030P and the second pixel signal (the Ra signal) generated in the second pixel 2031P, and generates a blood volume image signal. The blood volume image signal is a signal for displaying a blood volume image. The blood volume image is a color image on which information on the blood volume is superimposed. The memory 302 stores information required for calculating the blood volume, in addition to the information required for calculating the oxygen saturation.

The visible light image signal, the oxygen saturation image signal, and the blood volume image signal generated by the operation device 31 are output to the monitor 40. The monitor 40 displays the visible light image based on the visible light image signal, the oxygen saturation image based on the oxygen saturation image signal, and the blood volume image based on the blood volume image signal. For example, the monitor 40 displays the visible light image, the oxygen saturation image, and the blood volume image side by side. Alternatively, the monitor 40 displays an image selected from these images by a user. The configuration shown in FIG. 13 is the same as that shown in FIG. 1, except for the aforementioned points.

A calculation method of the blood volume will be described. The operation device 31 (the blood volume image generation unit 303) calculates the first intensity ratio for each of the plurality of first pixels 2030P. The first intensity ratio is an intensity ratio (Ra/G) of the Ra signal and the G signal. The Ra signal generated in one second pixel 2031P is used to calculate the first intensity ratio of four first pixels 2030P corresponding to the second pixel 2031P.

The memory 302 stores information indicating a correlation between the first intensity ratio and the blood volume. In the correlation, the blood volume increases with an increase in the first intensity ratio. The operation device 31 (the blood volume image generation unit 303) reads the information on the blood volume from the memory 302. The operation device 31 (the blood volume image generation unit 303) collates the calculated first intensity ratio and the information read from the memory 302. In this way, in the first pixel 2030P, the blood volume corresponding to the first intensity ratio is decided. The blood volume is calculated for each of the plurality of first pixels 2030P. The blood volume may be calculated for only a first pixel 2030P corresponding to a vessel region in an angle of view.

In the wavelength band B10 of light passing through the optical filter 2033, the light absorption coefficient of the oxidized hemoglobin is small. Therefore, a change in the Ra signal according to a change in the blood volume is small. However, the Ra signal is changed depending on optical conditions. For example, the optical conditions include a distance between the imager 203 and the object 50, an intensity of light emitted from the light source 100, and the like. On the other hand, in the wavelength band B10, the light absorption coefficient of the reduced hemoglobin is large. Therefore, a change in the G signal according to a change in the blood volume is large and the G signal is changed depending on the optical conditions. The operation device 31 (the oxygen saturation image generation unit 301) can calculate a blood volume not affected by the optical conditions by calculating the intensity ratio (R/G) of the R signal and the G signal.

The operation device 31 (the blood volume image generation unit 303) generates the blood volume image signal on the basis of the R signal, the G signal, the B and the blood volume. For example, the operation device 31 (the blood volume image generation unit 303) multiplies only the B signal by a gain larger than 1 in the first pixel 2030P in which the blood volume is smaller than a reference value 6. The operation device 31 (the blood volume image generation unit 303) may multiply only the B signal by the gain larger than 1 in the first pixel 2030P in which the blood volume is smaller than the reference value δ and is equal to or more than a reference value ε. The reference value ε is smaller than the reference value δ. The reference value δ is a threshold value for determining a region Where the blood volume is small. The reference value δ is a threshold value for determining a region Where there is no blood. The reference value δ and the reference value ε may be values Which can be designated by a user. Instead of multiplying only the B signal by the gain, a gain may be multiplied to the R signal, the G signal, and the B signal and the gain multiplied to the B signal may be larger than a gain multiplied to the R signal and the G signal. The blood volume image signal includes the R signal, the G signal, and the B signal of each first pixel 2030P.

In a region where the blood volume is smaller than the reference value δ and is equal to or more than the reference value s, since the gain multiplied to the B signal is large, the region has a bluish color in the blood volume image. In general, in a color in a living body, a red component is large and a blue component is small. Therefore, the bluish region is conspicuous in the blood volume image. It this way, after a cancerous region is resected, a doctor can determine the volume of blood flowing through a living body tissue. For example, the living body tissue includes the stomach, the large intestine and the like. When the blood volume is small, it is probable that the tissue will necrose. After a cancerous region is resected, a doctor can determine whether to suture cut blood vessels, on the basis of the blood volume. On the other hand, in the first pixel 2030P in which the blood volume is equal to or more than the reference value δ or is smaller than the reference value ε, no gain is multiplied to the B signal, the G signal and the R signal. In the blood volume image, there is no change in color tone of the region where the blood volume is equal to or more than the reference value γ.

As described above, the plurality of first pixels 2030P include the G pixel Pg1 that generates the first pixel signal based on the green light. The operation device 31 (the blood volume image generation unit 303) calculates the blood volume in each first pixel 2030P included in the plurality of first pixels 2030P on the basis of the first pixel signal (the G signal) generated in the G pixel Pg1 and the second pixel signal (the Ra swig). The operation device 31 (the blood volume image generation unit 303) processes the first pixel signal in order to emphasize and display a region, where the blood volume is smaller than a first threshold value (a reference value δ), in an image based on the first pixel signal generated in the plurality of first pixels 2030P. In this way, in the image based on the first pixel signal, the region, where the blood volume is smaller than the first threshold value, is conspicuous.

The operation device 31 (the blood volume image generation unit 303) processes the first pixel signal (the B signal) in order to emphasize and display only a region, where the blood volume is smaller than the first threshold value (the reference value δ) and is equal to or more than a second threshold value (the reference value ε), in an image based on the first pixel signal (the R signal, the G signal, and the B generated in the plurality of first pixels 2030P. The second threshold value is smaller than the first threshold value. In this way, in the image based on the first pixel signal the region where the blood volume is smaller than the first threshold value and is equal to or more than the second threshold value, is conspicuous.

While preferred embodiments of the invention have been described and shown above, it should be understood that these are exemplars of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscope system comprising:
a light source configured generate illumination light including visible light;
an imaging device configured to image reflected light of the illumination light, irradiated to an object from the light source; and
an operation device,
wherein the imaging device comprises:
a first substrate having a plurality of first pixels;
a second substrate stacked on the first substrate and having a plurality of second pixels; and
an optical filter disposed between the first substrate and the second substrate and configured to allow only light having passed through the first substrate and having a predetermined wavelength band for calculating an oxygen saturation to pass therethrough,
at all wavelengths included in the predetermined wavelength band, a light absorption coefficient of oxidized hemoglobin is larger than a light absorption coefficient of reduced hemoglobin, or at all the wavelengths included in the predetermined wavelength band, the light absorption coefficient of the oxidized hemoglobin is smaller than the light absorption coefficient of the reduced hemoglobin,
the reflected light is incident on the plurality of first pixels,
the light having passed through the first substrate and the optical filter is incident on the plurality of second pixels,
the first pixel included in the plurality of first pixels are configured to generate a first pixel signal based on the light incident on the first pixel,
the second pixel included in the plurality of second pixels are configured to generate a second pixel signal based on the light incident on the second pixel, and
the operation device is configured to calculate the oxygen saturation on the basis of the first pixel signal and the second pixel signal.

2. The endoscope system according to claim 1, wherein the predetermined wavelength band is a wavelength band in which a wavelength is equal to or more than 500 nm.

3. The endoscope system according to claim 2, wherein the predetermined wavelength band is included in a wavelength band in which a wavelength is 500 nm to 750 nm and has a width equal to or less than 100 nm.

4. The endoscope system according to claim 2, wherein the light source is configured to generate the illumination light including light having the predetermined wavelength band, in addition to the visible light, and
the predetermined wavelength band is included in a wavelength band in which a wavelength is 800 nm to 900 nm and has a width equal to or less than 100 nm.

5. The endoscope system according to claim 1, wherein the operation device is configured to generate a corrected pixel signal by subtracting a signal based on the second pixel signal from the first pixel signal, and calculate the oxygen saturation on the basis of the corrected pixel signal and the second pixel signal.

6. The endoscope system according to claim 1, wherein the plurality of first pixels include a G pixel that is configured to generate the first pixel signal based on green light, and
the operation device is configured to calculate a blood volume in each first pixel included in the plurality of first pixels on the basis of the first pixel signal generated in the G pixel and the second pixel signal, and process the first pixel signals in order to emphasize and display a region, where the blood volume is smaller than a first threshold value, in an image based on the first pixel signals generated in the plurality of first pixels.

7. The endoscope system according to claim 6, wherein the operation device is configured to process the first pixel signals in order to emphasize and display only a region, where the blood volume is smaller than the first threshold value and is equal to or more than a second threshold value, in the image based on the first pixel signals generated in the plurality of first pixels, the second threshold value being smaller than the first threshold value.

8. The endoscope system according to claim 1, wherein the operation device is configured to calculate the oxygen saturation in each first pixel included in the plurality of first pixels and process the first pixel signals in order to emphasize and display a region, where the oxygen saturation is smaller than a predetermined threshold value, in the image based on the first pixel signals generated in the plurality of first pixels.
